(19) United States
(12) Patent Application Publication
(10) Pub. No.: US 2018/0161377 A1
(43) Pub. Date: Jun. 14, 2018

(54) METHOD OF TREATING NEUROLOGICAL DISORDERS WITH STEM CELL THERAPY

(71) Applicant: NEONEURON LLC, San Antonio, TX (US)

(72) Inventor: Marcel M Daadi, Palo Alto, CA (US)

(73) Assignee: NEONEURON LLC, San Antonio, TX (US)

(21) Appl. No.: 15/836,849

(22) Filed: Dec. 9, 2017

Related U.S. Application Data
(60) Provisional application No. 62/432,544, filed on Dec. 9, 2016.

(51) Int. Cl. A61K 35/30; A61K 9/10; A61K 9/00; A61P 25/16; A61P 25/24; A61P 25/18; A61P 25/30; A61P 25/00; A61P 25/28; A61P 25/14; C12N 5/0797; C12N 5/0793

(52) U.S. Cl. CPC A61K 35/30; C12N 2500/32; A61K 9/0085; A61P 25/16; A61P 25/24; A61P 25/18; A61P 25/30; A61P 25/00; A61P 25/28; A61P 25/14; C12N 5/0623; C12N 5/0619; C12N 2501/11; C12N 2501/115; C12N 2506/03; C12N 2506/02; C12N 2500/38; C12N 2501/15; C12N 2501/415; C12N 2500/30; A61K 9/10

(57) ABSTRACT

A method for isolating self-renewable stem cells from pluripotent stem cells (embryonic or induced) by treating the pluripotent stem cells with a combination of epidermal growth factor and basic growth factor, wherein upon treatment, the pluripotent stem cells differentiate into self-renewable neural stem cells. The self-renewable stem cells may be further induced to dopaminergic neurons through treatment with dopaminergic inducing media. The dopaminergic neurons may be administered in a cell suspension, alone or in combination with purified glial cells, directly into the brain tissue of patients suffering from neurological disorders.

### CROSS-REFERENCE TO RELATED APPLICATIONS

Pursuant to 35 U.S.C. § 119(e), this application claims priority to and the benefit of U.S. Provisional Patent Application No. 62/432,544 filed on Dec. 9, 2016, the disclosure of which is incorporated by reference in its entirety herein.

### TECHNICAL FIELD

The present invention relates generally to stem cell therapy. More specifically, the present invention relates to a method of isolating self-renewable neural stem cells from pluripotent sterns cells and inducing the self-renewable stem cells to form dopaminergic (DA) neurons, wherein the self-renewable stem cells and/or the DA neurons are used for treating neurological disorders.

### BACKGROUND OF THE INVENTION

The degeneration and death of DA neurons in the midbrain is the hallmark of Parkinson's disease (PD), a devastating movement disorder that manifests with tremor, bradykinesia and rigidity. Over one million persons currently suffer from PD in the United States and there are approximately 60,000 newly diagnosed cases every year. The cost for treating PD is estimated at $27 billion per year. Obeso et al., Trends in Neuroscience 23(10):S2-7 (2000). Levodopa (L-DOPA), the mainstay pharmacotherapy for treating PD, produces effective relief of motor symptoms in the early stages of PD. As the disease progresses, the nigrostriatal dopaminergic terminals degenerate and consequently the conversion of L-DOPA to dopamine becomes inefficient and the dose required to control the PD symptoms must increase. The increased L-DOPA dosage leads to the development of abnormal involuntary movements or dyskinesia. Obeso et al., supra. Subsequently, there is a significant decline of the health and wellbeing of PD patients.

Proof of principal that implantation of DA neurons into the putamen of PD patients restores dopaminergic neurotransmission and improves L-DOPA pharmacotherapy has been clinically demonstrated using human fetal mesencephalic (MS) tissue. Mendez et al., Nature Medicine 14(5):507-509 (2008). Due to the method of tissue preparation, study design, and cell delivery, some clinical studies led to serious side effects in some patients. Freed et al., The New England Journal of Medicine 344(10):710-719 (2001). Further, because the procedure requires tissue from several aborted fetal brains to obtain the DA cells required to treat a single patient, the ethical issues and the impracticality associated with the procedure makes it highly unlikely that it could ever be developed into a safe product that could meet the large demand for the product.

There remains a need in the art for an effective and efficient method of consistently producing an unlimited supply of purified neural stem cells (NSCs) and DA neurons for various applications.

### SUMMARY OF THE INVENTION

The present invention overcomes the need in the art by providing a method for the isolation of self-renewable NSCs from somatic tissue and from pluripotent stem cells and the differentiation of the self-renewable NSCs into DA neurons with a glial secreted medium.

In one embodiment, the present invention is directed to a method for isolating a population of neural stem cells from pluripotent stem cells comprising the steps of: (a) obtaining pluripotent stem cells from embryonic stem cells obtained from an organism or inducing pluripotent stem cells from any somatic cell of a non-embryonic organism; and (b) treating the pluripotent stem cells with epidermal growth factor (EGF) in a concentration in the range of about 10 ng/mL to about 100 ng/mL and basic fibroblastic growth factor (bFGF) in a range of about 10 ng/mL to about 100 ng/mL, wherein the pluripotent stem cells differentiate into self-renewable neural stem cells. In another embodiment, the EGF and bFGF are both in a range of about 10 ng/mL to about 50 ng/mL. In a further embodiment, the EGF and the bFGF are both in a range of about 10 ng/mL to about 20 ng/mL.

In another embodiment, the pluripotent stem cells are treated with retinoic acid having a molar concentration in a range of about 0.1 μM to about 10 μM. In a further embodiment, the neural stem cells are treated with retinoic acid within one week of isolation from the pluripotent stem cells. In another embodiment, the self-renewable neural stem cells are passaged on a weekly basis.

In another embodiment, the self--renewable neural stem cells express at least one neural precursor marker. In a further embodiment, the at least one neural precursor marker is selected from the group consisting of nestin, vimentin, prominin-1, and Sox-2.

In another embodiment the present invention is directed to treating a neurological disorder comprising administering a cell suspension of the self-renewable neural stem cells to a patient suffering from the neurological disorder. In a further embodiment, the neurological disorder is selected from the group consisting of Alzheimer's disease, stroke, traumatic brain injury, amyotrophic lateral sclerosis, spinal cord injury, and Huntington's disease,

In another embodiment, the present invention is directed to a method for inducing dopaminergic neurons from neural stem cells comprising treating a culture of the self-renewable neural stem cells of the present invention with dopaminergic inducing media comprising at least one glial secreted soluble factor, wherein dopaminergic inducing media causes differentiation the self-renewable neural stem cells to differentiate into dopaminergic neurons expressing tyrosine hydroxylase. In a further embodiment, the dopaminergic inducing media further comprises bFGF, ascorbic acid, forskolin, and optionally cAMP and/or retinoic acid. In another embodiment, the at least one glial secreted soluble factor is present in the dopaminergic inducing media in a concentration ranging from about 25% wt/vol to about 75% wt/vol. In a further embodiment, the bFGF is present in the dopaminergic inducing media in a concentration ranging from about 10 ng/ml to about 100 ng/ml. In another embodiment, the ascorbic acid is present in the dopaminergic inducing media in a molar concentration of about 1 μM to about 1 mM. In a further embodiment, the forskolin is present in the dopaminergic inducing media in a molar concentration of about 1 μM to about 1 mM. In another embodiment, the at least one glial secreted soluble factor is a TFG-β family member that activates a TFG-β signaling, pathway. In a further embodiment, the TFG-β signaling pathway is a Wnt/β-catenin pathway.

In another embodiment, the culture of the self-renewable neural stem cells are further treated with an extracellular matrix or substrate selected from the group consisting of decellularized glial cells, decellularized neural cells, decellularized non-neuronal cells, poly-L-ornithine, fibronectin, and at least one laminin family member. In a further embodiment, the decellularized non-neuronal cells are choroid plexus cells. In another embodiment, the poly-L-ornithine is present in the extracellular matrix or substrate in a concentration of about 1 ng/ml to about 50 μg/ml. In a further embodiment, the fibronectin is present in the extracellular matrix or substrate in a concentration of about 1 ng/ml to about 50 μg/ml. In another embodiment, the at least one laminin family member is laminin-521 and/or laminin-511, wherein the laminin-521 and/or laminin-511 is present, individually or in combination, in the extracellular matrix or substrate in a concentration of about 1 ng/ml to about 50 μg/ml. In a further embodiment, the extracellular matrix or substrate are added to the culture of self-renewable neural stem cells prior to treatment with the dopaminergic inducing media. In another embodiment, the extracellular matrix or substrate is a solubilized mixture of protein added to the culture of self-renewable neural stem cells. In a further embodiment, the extracellular matrix or substrate coats a vessel upon which the self-renewable neural stem cells are cultured. In another embodiment, the extracellular matrix or substrate is a solubilized mixture of protein added to the dopaminergic inducing media. In a further embodiment, the culture of the self-renewable neural stem cells is further treated with exosomes.

In another embodiment, the present invention is directed to a method of treating a patient suffering from a neurological disorder associated with a loss or dysfunction of dopaminergic neurons comprising administering a cell suspension comprising the dopaminergic neurons of the present invention to the patient, wherein the dopaminergic neurons improve symptoms of the disease. In a further embodiment, the neurological disorder is selected from the group selected from Parkinson's disease, trauma, bipolar disorder, depression, addiction, and schizophrenia. In another embodiment, the cell suspension of dopaminergic neurons further comprises purified glial cells. In a further embodiment, the purified glial cells are selected from the group consisting of astrocytes, oligodendrocytes, and microglia. In another embodiment, the purified glial cells are genetically engineered or processed to secrete neurotrophic factors and/or immunomodulatory cytokines. In a further embodiment, the neurotrophic factors and/or immunomodulatory cytokines are selected from the group consisting of insulin growth factor-1, interleukin-10, and interleukin-13. In another embodiment, the dopaminergic/glial cell suspension has a proportion of dopaminergic neurons ranging from 0.01% to 99.99%. In a further embodiment, the cell suspension of dopaminergic neurons further comprises a decellularized extracellular matrix derived from one or more glial cell cultures.

In another embodiment, the neurological disorder is Parkinson's disease and the dopaminergic cell suspension of the present invention is implanted into basal ganglia forebrain areas of a patient with Parkinson's disease. In a further embodiment, the cell suspension is implanted into putamen and/or caudate areas of the basal ganglia of a patient with Parkinson's disease. In another embodiment, the cell suspension is implanted into striatum forebrain areas of a patient with Parkinson's disease. In a further embodiment, the cell suspension is implanted into substantia nigra midbrain areas of a patient with Parkinson's disease.

Additional aspects and embodiments of the invention will be provided, without limitation, in the detailed description of the invention that is set forth below.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1A is a photograph showing three dimensional organoid morphology of neural stem cells (NSCs) isolated from induced pluripotent stem cells (iPSC) grown in a suspension culture. FIG. 1B is a photograph showing two-dimensional morphology of NSCs derived from iPSCs. FIG. 1C is a photograph showing three dimensional organoid morphology of NSCs isolated from cadaveric brain tissue and grown in a suspension culture. FIG. 1D is a graph showing the long-term growth stability of NSCs isolated from iPSCs over a period of 90 days.

FIG. 2A is a phase contrast image showing the differentiation of NSCs into dopaminergic (DA) neurons. FIG. 2B is a photograph showing that NSCs respond to dopamine-inducing factors and express the tyrosine hydroxylase marker for DA neurons. FIG. 2C is a graph showing that quantitative-polymerase chain reaction (Q-PCR) analysis performed on NSCs for selected transcription factors confirms the dopaminergic identity of the NSCs.

FIG. 3 is a schematic showing the synergistic effect of glial soluble factors with bFGF on the activation of the bFGF and TGF-β signaling molecules in the cell cytoplasm and nucleus of dopamine-induced NSCs.

FIG. 4 is a graph showing that Q-PCR analysis of the mRNA isolated from dopamine-induced NSCs demonstrate enhanced expression and activation of genes belonging to the TGF-β family of growth factors.

FIG. 5 is a schematic showing the synergistic effect of glial soluble factors with bFGF on the activation of the Wnt/β-catenin TGF-β signaling pathway in the cell cytoplasm and nucleus of dopamine-induced NSCs.

FIG. 6 is a graph showing that Q-PCR analysis of mRNA isolated from dopamine-induced NSCs demonstrate enhanced expression and activation of genes belonging to the Wnt/β-catenin signaling pathway.

FIG. 7 is a schematic showing the activation of the Sonic hedgehog Signaling Wnt/β-catenin signaling pathway in the cell cytoplasm and nucleus of dopamine-induced NSCs.

FIG. 8 is a graph showing the increase in expression and activation of genes associated with dopaminergic cell maturation and function in dopamine-induced NSCs relative to control (non-dopamine-induced) NSCs.

FIG. 9 is a heat map representation of significantly upregulated genes in dopamine-induced NSCs relative to control (non-dopamine-induced) NSCs. The fold of increase in indicated within the color bars.

FIG. 10A is a photograph showing immunostaining differentiated iPSC-derived NSCs with the DA neuron cell marker tyrosine hydroxylase (TH, red). FIG. 10B is a graph showing the dopamine released (in ng/mL) from dopamine-induced NSCs in response to 15 minutes of KCl stimulation quantified by ELISA.

FIG. 11A is a graph showing the results of an apomorphine-induced rotational test in rats with induced Parkinsonian symptoms (Vehicle) versus apomorphine-induced Parkinsonian rats that underwent a DA neural cell implant (DA TX). FIG. 11B is a graph showing that a Parkinsonian rat showed improved use of its contralateral forelimb (Contra) following a DA neural cell implant (DA TX).

FIG. 12A is a low power photomicrograph of a frontal brain tissue section across the forebrain of a Parkinsonian rat showing graphs of DA neurons (in the putamen and caudate of the striatum) immunolabeled with anti-TH (arrow). FIG. 12B is low power photomicrograph of another section of the frontal brain tissue shown in FIG. 12A.

### DETAILED DESCRIPTION OF THE INVENTION

Set forth below is a description of what are currently believed to be preferred embodiments of the claimed invention. Any alternates or modifications in function, purpose, or structure are intended to be covered by the claims of this application. As used in this specification and the appended claims, the singular forms “a,” “an,” and “the” include plural referents unless the context clearly dictates otherwise. The terms “comprises” and/or “comprising,” as used in this specification and the appended claims, specify the presence of stated features, integers, steps, operations, elements, and/or components, but do not preclude the presence or addition of one or more other features, integers, steps, operations, elements, components, and/or groups thereof. All references and literature cited in this section as well as in the Background of the Invention above and the Experimental Section that follows are incorporated in their entireties.

As used herein, the term “pluripotent stem cells” refer to cells that have the capability to give rise to any cell type of the body. These cells have an unlimited ability to proliferate and differentiate into specialized functional cells. They can be derived from the inner cell mass of a blastocyst embryonic stage. In this case they are called embryonic stem cells. Pluripotent stem cells can also be induced from any somatic cell of a non-embryonic organism, such as blood or skin cells using a variety of genetic or chemical techniques. In this case the pluripotent stem cells are called induced pluripotent stem cells (iPSCs). Within the context of the claimed invention, the term “pluripotent stem cell” is meant to include both embryonic stem cells and iPSCs.

As used herein, the term “organism” is meant to include any human or non-human animal mammal.

As used herein, the term “brain tissue” is meant to include all portions of a human or non-human animal brain.

As used herein, the term “patient” is meant to refer to any human or non-human animal species that is suffering from one or more of the neurological conditions referenced herein.

The present invention describes method for the isolation and perpetuation of a specific population of self-renewable neural stem cells (NSCs) derived from pluripotent stem cells or from brain tissue and method for differentiating the NSCs into dopaminergic (DA) neurons. The pluripotent stem cells from which the NSCs are isolated are embryonic stem cells or iPSCs that are generated from blood cells, skin biopsy, or any other type of somatic cell taken from any person or from the patient. Somatic cells may be rendered pluripotent through one or more techniques including without limitation, the Yamanaka gene combination (Okita et al., Nature Protocols 5(3):418-428 (2010)), application of nucleic acid mixtures, application of tissue culture conditions, application of certain proteins, application of specific chemicals, and combinations thereof. The sources of the pluripotent stem cells may include, without limitation, humans, nonhuman primates, swine, and/or rodents.

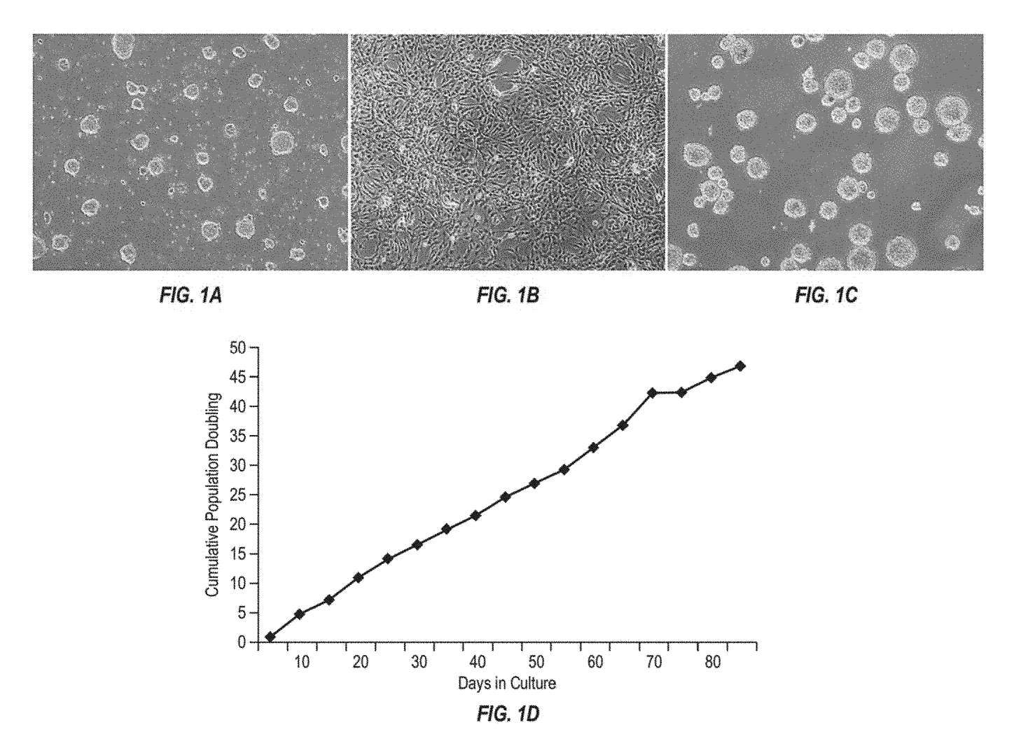

in one embodiment, the NSCs are isolated from iPSCs (FIGS. 1A and 1B) and are able to generate all cell types of the nervous system. In another embodiment, the NSCs are isolated from brain tissue (FIG. 1C). The NSCs described herein are capable of generating an unlimited supply of neural cells for biological and therapeutic use (FIG. 1D). FIG. 1D shows the significant growth rate (cumulative population doubling) of self-renewable NSCs isolated from iPSCs over a period of 90 days in culture. The self-renewable NSCs described express neural precursor markers, which include without limitation, the neural precursor nestin, vimentin, prominin-1, and Sox-2. The NSCs are isolated from pluripotent sterns cells obtained from embryonic stem cells from an organism or from iPSCs from any somatic cell of a non-embryonic organism by treating the pluripotent stem cells with epidermal growth factor (EGF) and basic fibroblastic growth factor (bFGF), the treatment of which induces the pluripotent stem cells to differentiate into NSCs. The EGF and bFGF are each individually added to the pluripotent stem cells are in a range of about 10 ng/mL to about 100 ng/mL. More specific ranges of the EGF and bFGF are about 10 ng/mL to about 50 ng/mL each or even more specifically, about 10 ng/mL to about 20 ng/mL. In addition to the EGF and bFGF, the pluripotent stem cells may be further treated with retinoic acid. In one embodiment, the retinoic acid, which may be added to the pluripotent stem cells in a molar concentration ranging from about 0.1 μM to about 10 μM. In other embodiment, the retinoic acid is added to the NSCs within one week of isolation from the pluripotent stem cells. Once the pluripotent stem cells have differentiated into the self-renewable NSCs, the NSCs may be passaged on a weekly basis.

The NSCs described herein may be used to treat a neurological disorder by administering a cell suspension of the NSCs to a patient suffering from a neurological disorder. The NSC cell suspension may be administered to the patient directly in the site of the neurological disorder. For example, the cell suspension of the NSCs may be administered directly into the brain or spinal cord of the patient. Examples of neurological disorders that may be treated with the NSCs of the present invention include, without limitation, Alzheimer's disease, stroke, traumatic brain injury, amyotrophic lateral sclerosis (ALS), spinal cord injury, and Huntington's disease.

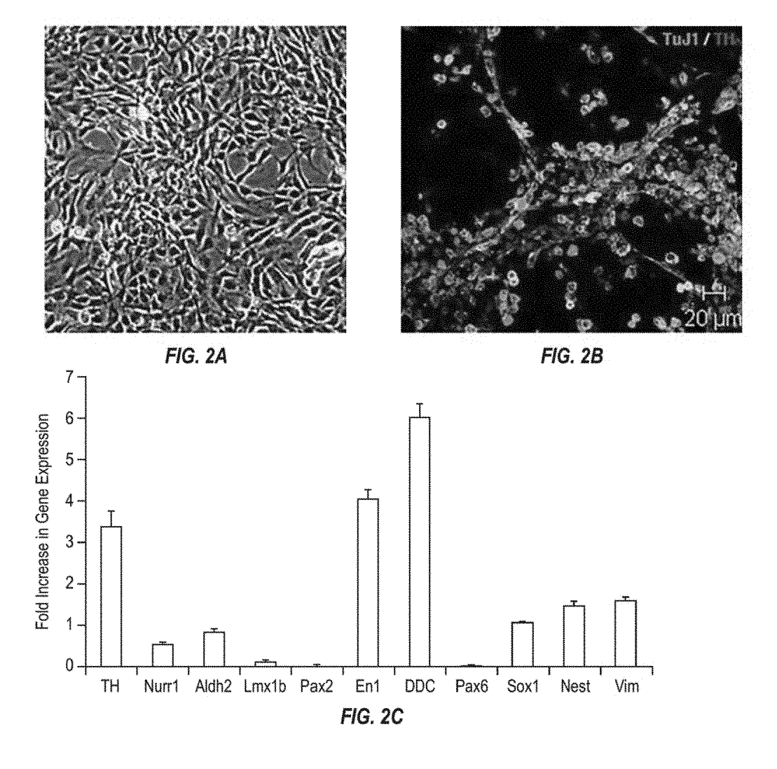

In another embodiment, isolated and expanded NSCs are able to produce DA neurons when exposed with media comprising at least one glial secreted soluble factor (Example 1), the latter of which are obtained from glial cells. This media, which is referred to herein as “dopaminergic-inducing media” may include additional factors such as, basic fibroblast growth factors (bFGF), ascorbic acid, forskolin, and optionally dibutryl-cyclic AMP (cAMP) and/or retinoic acid. FIGS. 2A. and 2B show DA neurons induced from NSCs using dopaminergic inducing media. The at least one glial secreted soluble factor is present in the dopaminergic inducing media in a concentration ranging from about 25% wt/vol. to about 75% wt/vol., more specifically from about 50% wt/vol to about 75% wt/vol. Where the dopaminergic inducing media includes bFGF, the bFGF is present in the dopaminergic inducing media in a concentration ranging from about 10 ng/mL to about 100 ng/mL, more specifically from about 10 ng/mL to about 50 ng/mL. Where the dopaminergic inducing media includes ascorbic acid, the ascorbic acid is present in the dopaminergic inducing media in a molar concentration ranging from about 1 μM to about 1 mM, more specifically from about 100 μM to about 200 μM. Where the dopaminergic inducing media includes forskolin, the forskolin is present in the dopaminergic inducing media in a molar concentration ranging from about 1 μM to about 1 mM, more specifically from about 10 μM about 60μM. Where the dopaminergic inducing media includes cAMP, the cAMP is present in the dopaminergic inducing media in a molar concentration ranging from about 1 mM to about 50 mM, more specifically, about 2 mM to about 20 μM. Where the dopaminergic inducing media include retinoic acid, the retinoic acid is present in the dopaminergic inducing media in a molar concentration ranging from about 1 μM to about 20 μM, more specifically from about 0.1 μM and 10 μM.

The dopaminergic phenotype of the DA neurons described herein may be enhanced by exposure of NSCs to extracellular matrices or substrates prior to treatment with the dopaminergic inducing media. The extracellular matrices or substrates may be isolated from decellularized glial cells, decellularized neural cells, decellularized non-neural cell cultures (such as choroid plexus cells), poly-L-ornithine, fibronectin, collagen, and/or at least one laminin family member. Where the extracellular matrix or substrate includes poly-L-ornithine, the poly-L-ornithine is present in the extracellular matrix or substrate in a concentration of about 1 ng/mL to about 50 μg/mL, more specifically 1 μg/mL to about 50 μg/mL. Where the extracellular matrix or substrate includes fibronectin, the fibronectin is present in the extracellular matrix or substrate in a concentration of about 1 ng/mL to about 50 μg/mL, more specifically 1μg/mL to about 50 μg/mL. Where the extracellular matrix or substrate includes collagen, the collagen is present in the extracellular matrix or substrate in a concentration of about 1 ng/mL to about 50 μg/mL, more specifically 1 μg/mL to about 50 μg/mL. Where the extracellular matrix or substrate includes at least one laminin family member, the at least one laminin family member may be laminin-521 and/or laminin-511, which may be present, either individually or in combination, in the extracellular matrix or substrate in a concentration of about 1 ng/mL to about 50 μg/mL, more specifically 1 μg/mL to about 50 μg/mL. In one embodiment, the extracellular matrix or substrate may be added to the culture of self-renewable NSCs prior to treatment with the dopaminergic inducing media by adding the extracellular matrix or substrate as a soluble mix or proteins to the culture of NSCs. In another embodiment, the extracellular matrix or substrate is used to coat vessels upon which the NSCs are cultured prior to treatment with the dopaminergic inducing media. In a further embodiment, the extracellular matrix or substrate is added to the dopaminergic inducing media as soluble mix of proteins.

As shown in FIG. 2C, dopamine inducing treatments of NSCs induce the expression of genes that identify and determine DA neurons of the midbrain, the latter of which include without limitation, TH, Nurr1, Aldh2, Lmx1b, En1, DDC, Sox-1, Nest, and Vim. The dopaminergic identity of the dopamine-induced neurons may be confirmed by measuring the level of expression of genes that identify DA neurons using quantitative PCR (Q-PCR).

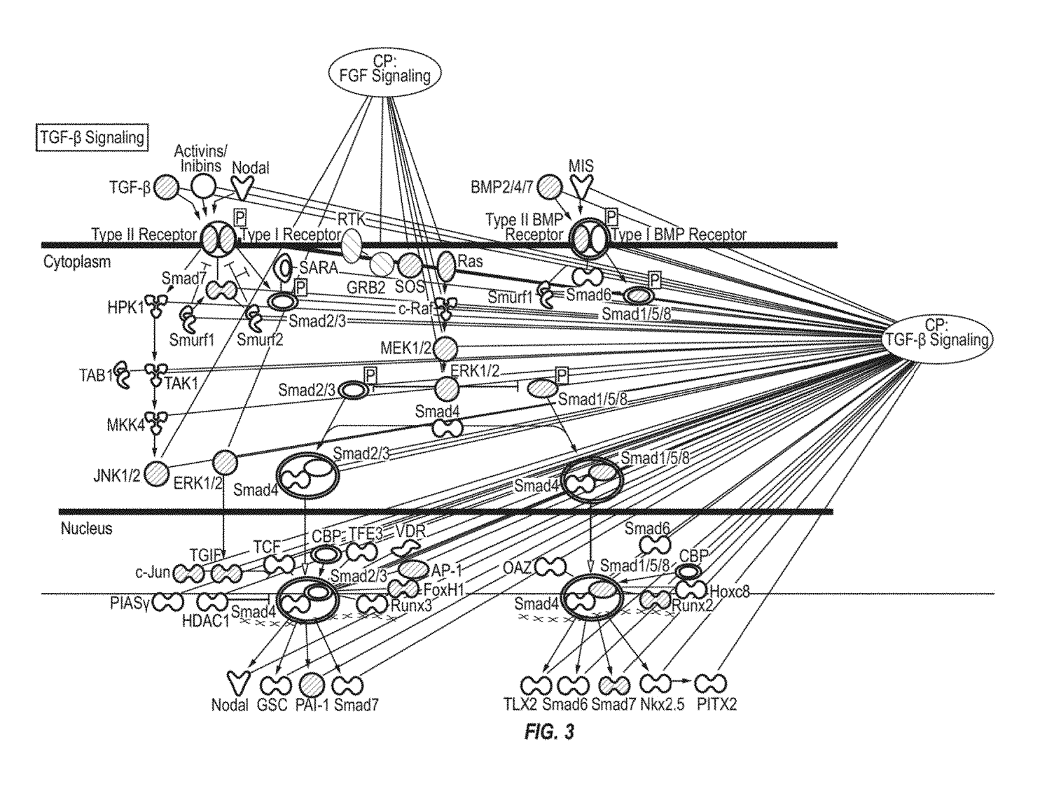

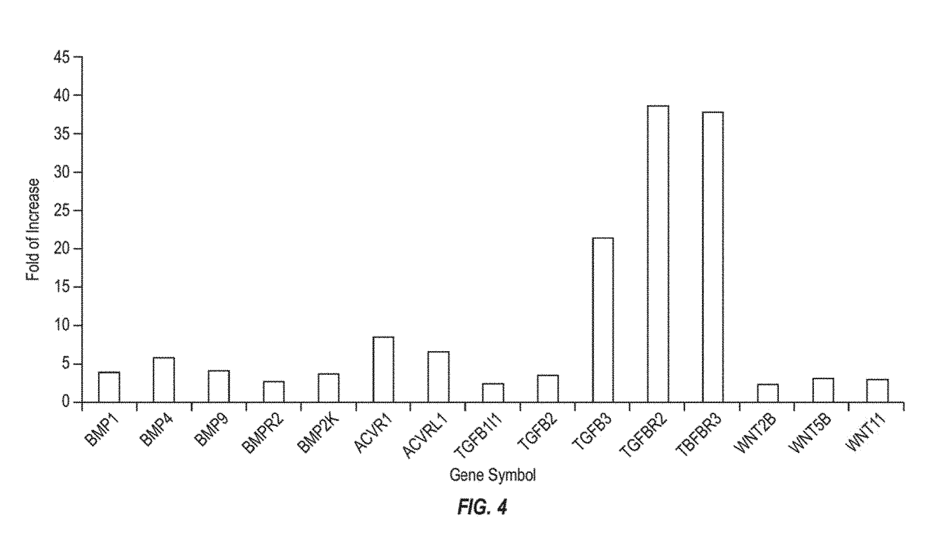

As shown in FIGS. 3 and 4, dopamine inducing treatments of NSCs activate the TGF-β signaling pathway in the NSCs as revealed by gene expression analysis. FIG. 3 demonstrates the synergistic effect of glial soluble factors with bFGF on the activation of bFGF and TGF-β signaling molecules in the cell cytoplasm and nucleus of dopamine-induced NSCs. As shown in FIG. 3, the intracellular targets include, but are not limited to, Smad family members, ERK family members, SOS, Ras, JNK, MEK, AP-1, FoxH1, TGIF, Runx2, c-Jun, and PAI-1 (FIG. 3). Cell surface receptors mediating the effects include, but not limited to, Type I/Type II TGF-β family members receptors and tyrosine kinase receptors (FIGS. 3 & 4). FIG. 4 shows that Q-PCR analysis of the mRNA isolated from dopamine-induced NSCs demonstrate enhanced expression and activation of genes belonging to the TGF-β family of growth factors.

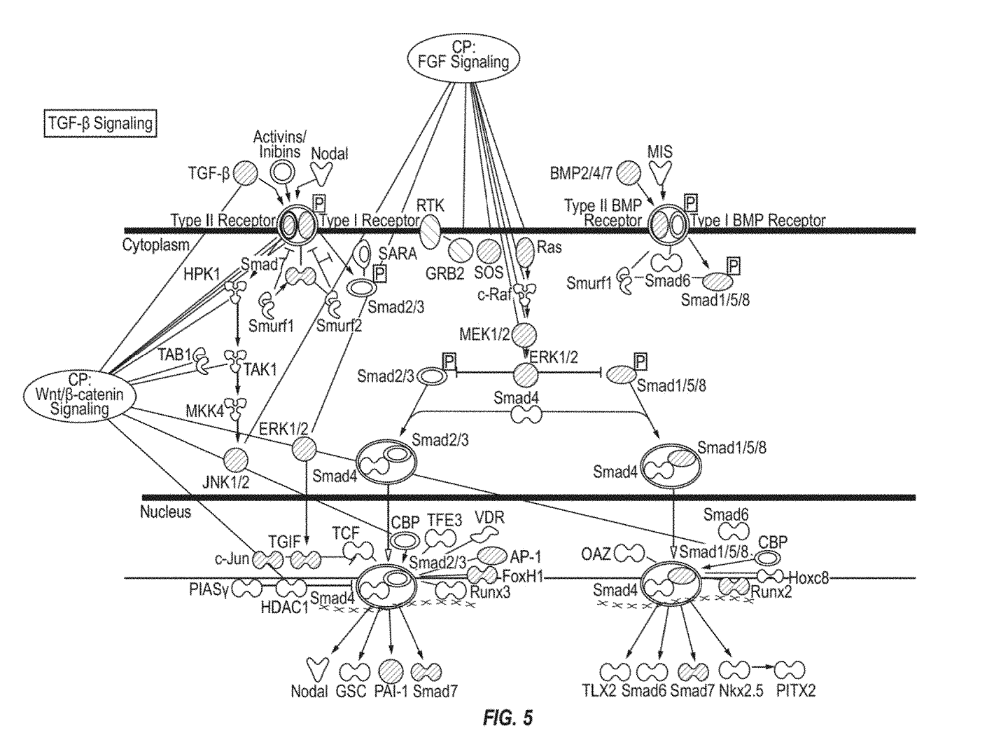

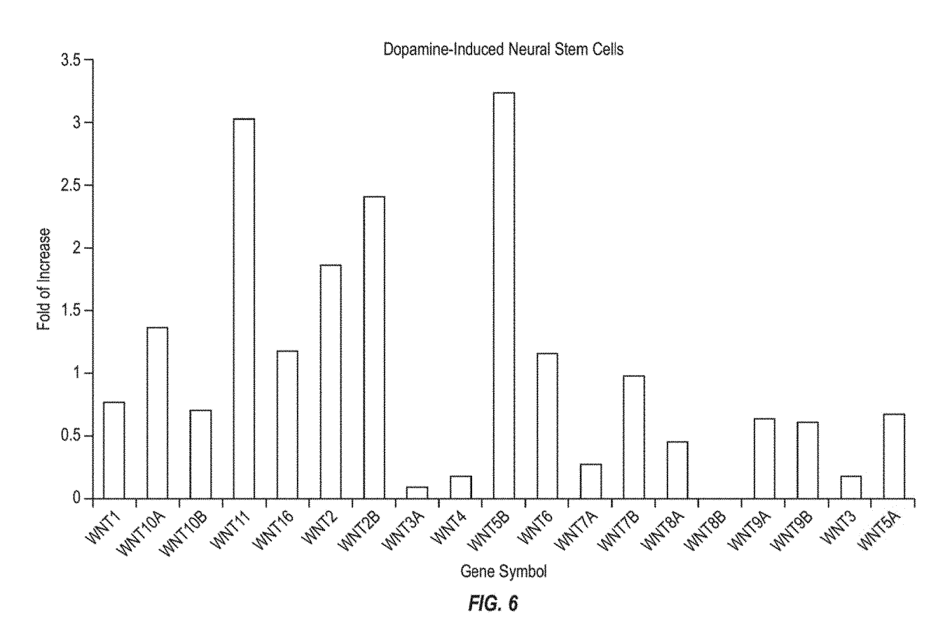

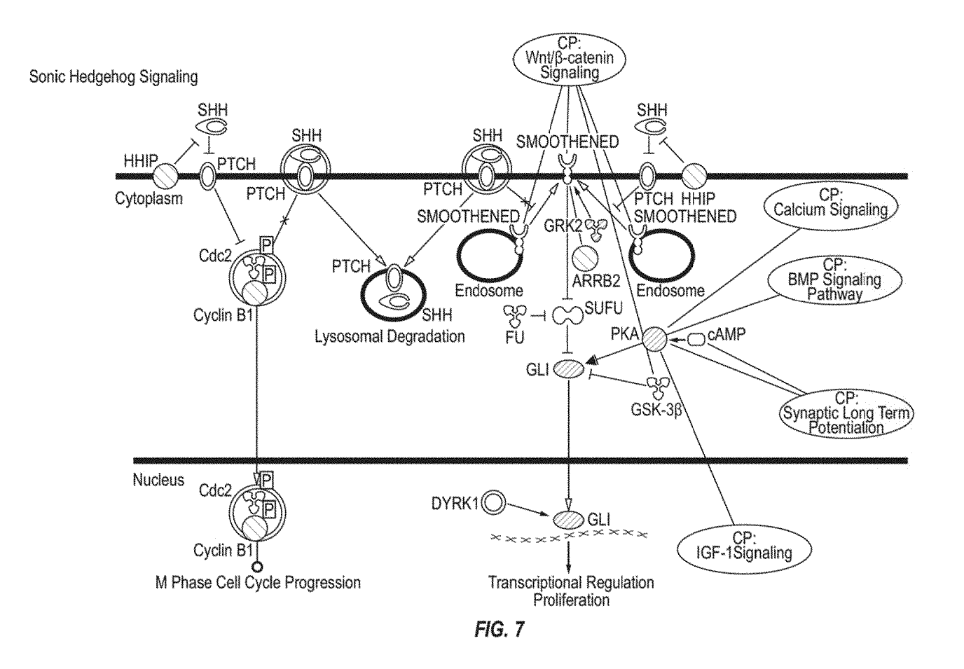

As shown in FIGS. 5 and 6, dopamine inducing treatments of NSCs activate the Wnt/β-catenin intracellular signaling pathway. FIG. 5 shows the synergistic effect of glial soluble factors with bFGF on the activation of the Wnt/β-catenin TGF-β signaling pathway in the cell cytoplasm and nucleus of dopamine-induced NSCs. The dopamine-inducing conditions that stimulate the Wnt pathway including, but are not limited to, Wnt-10, Writ-11, Wnt-16, Wnt-2, Wnt-5, Wnt-6 (FIG. 6). FIG. 6 shows that Q-PCR analysis of mRNA isolated from dopamine-induced NSCs demonstrate enhanced expression and activation of genes belonging to the Wnt/β-catenin signaling pathway. Other Wnt-β-catenin signaling pathways activates intracellular determinants include, but are not limited to, GRK2, PKA, GLI (FIG. 7). FIG. 7 shows the activation of the Sonic hedgehog Signaling Wnt/β-catenin signaling pathway in the cell cytoplasm and nucleus of dopamine-induced NSCs.

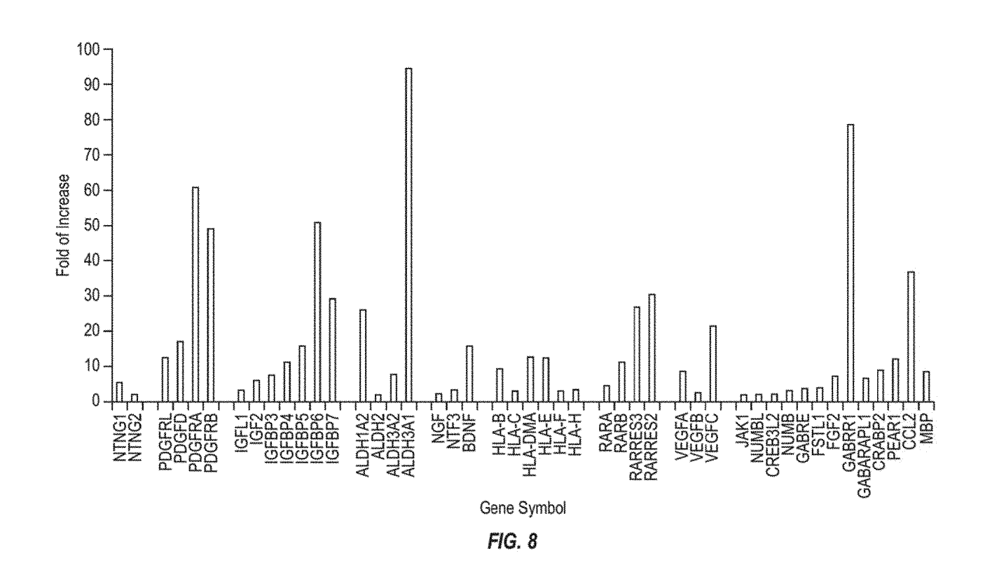

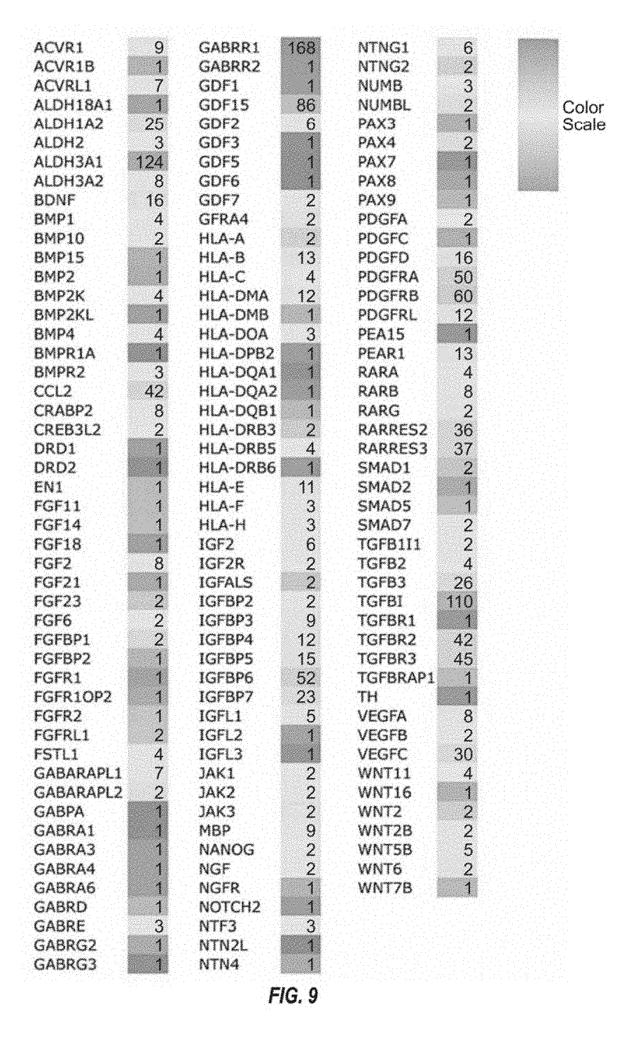

Genes upregulated during the induction of DA neurons in NSCs may include without limitation, ACVR1, ACVR1B, ACVRL1, ALDH18A1, ALDH1A2, ALDH2, ALDH3A1, ALDH3A2, BDNF, BMP1, BMP10, BMP2, BMP2K, BMP2KL, BMP4, BMPR1A, BMPR2, CCL2, CRABP2, CREB3L2, DRD1, DRD2, EN1, FGF11, FGF14, FGF18, FGF2, FGF21, FGF23, FGF6, FGFBP1, FGFBP2, FGFR1, FGFR1OP2, FGFR2, FGFRL1, FSTL1, GABARAPL1, GABARAPL2, GABPA, GABRA1, GABRA3, GABRA4, GABRA6, GABRD, GABRE, GABRG2,GABRG3, GBRR1, GABRR2, GDF1, GDF15, GDF2, GDF3, GDF5, GDF6, GDF7, GFRA4, HLA-A, HLA-B, HLA-C, HLA-DMA, HLA-DMB, HLA-DOA, HLA-DPB2, HLA-DQA1, HLA-DQA2, HLA-DQB1, HLA-DRB3, HLA-DRB5, HLA-DRB6, HLA-E, HLA-F, HLA-H, IGF2, IGF2R, IGFALS, IGFBP2, IGFBP3, IGFBP4, IGFBP5, IGFBP6, IGFBP7, IGFL1, IGFL2, IGFL3, JAK1, JAK2, JAK3, MBP, NANOG, NGF, NGFR, NOTCH2, NTF3, NTN2L, NTN4, NTNG1, NTNG2, NUMB, NUMBL, PAX3, PAX4, PAX7, PAX8, PAX9, PDGF, PDGFC, PDGFD, PDGFRA, PDGFRB, PDGFRL, PEA15, PEAR1, RARA, RARB, RARG, RARRES2, RARRES3, SMAD1, SMAD2, SMAD5, SMAD7, TGFB1I1, TGFB2, TGFB3, TGFBI, TGFBR1, TGFTBR2, TGFBR3, TGFBRAP1, TH, VEGFA, VEGFB, VEGFC, WNT11, WNT16, WNT2, WNT2B, WNT5B, WNT6, WINT7B, and combinations thereof (FIGS. 8 & 9). FIG. 8 shows the increase in expression and activation of genes associated with dopaminergic cell maturation and function in dopamine-induced NSCs relative to a control sample of non-dopamine-induced NSCs. FIG. 9 is a heat map representation of significantly upregulated genes in dopamine-induced NSCs relative to a control sample of non-dopamine-induced NSCs. The fold of increase of the dopamine-induced NSC samples versus the control samples is indicated within the color bars.

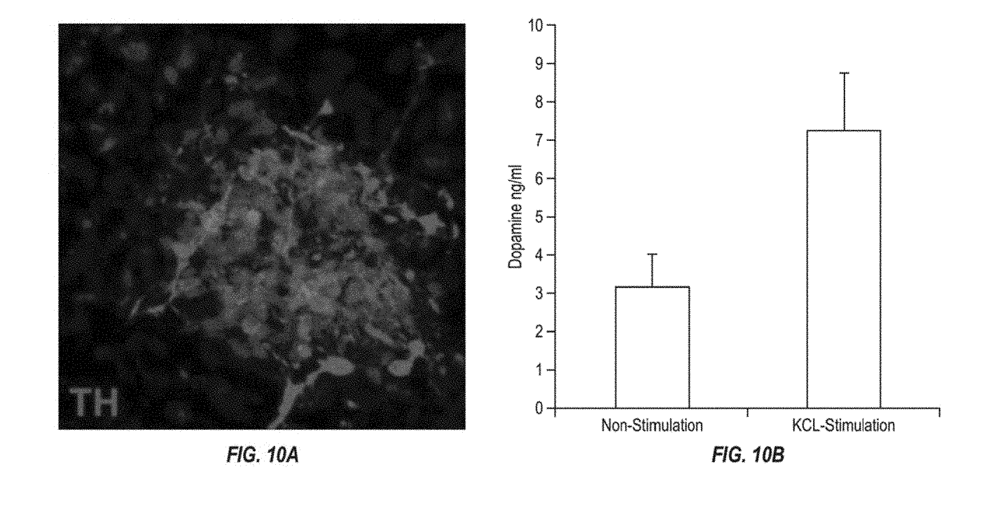

As shown in FIGS. 10A and 10B, secretion of dopamine by the DA neurons described herein is enhanced by the stimulation of cell culture with KCl. Glial-secreted factors include the TGF-β superfamily members. Within the context of the present invention, and while not intending to be limited by theory, the TGF-β superfamily members, which include Activin-A and BMP-2, are proteins that work synergistically with fibroblast growth factors to enhance the differentiation of the NSCs into DA neurons by inducing the expression of tyrosine hydroxylase (TH), the rate-limiting enzyme for the biosynthesis of the neurotransmitter dopamine and the key marker of DA neurons it NSC progeny (FIG. 10A). FIG. 10A shows that a cell culture of DA neurons induced from iPSC-derived NSCs secrete dopamine when immunostained with the TH cell marker (red). FIG. 10B shows that after 15 minutes of KCl stimulation, dopamine-induced NSCs showed an increase in dopamine release (in ng/mL) in comparison to non-stimulated dopamine-induced NSCs.

The DA neurons described herein may be used to treat neurological disorders caused by the loss or dysfunction of DA neurons through implantation of a suspension of the DA neurons described herein directly into the areas of the brains or organs of the patient that are defined by the loss or dysfunction of the DA neurons. Examples of such neurological disorders include without limitation, Parkinson's disease, trauma, bipolar disorder, depression, addiction, and schizophrenia.

The suspension of DA neurons used to treat the neurological disorders may further include purified glial cells. The glial cells may be derived from brain tissue or from neural stem cells by adding human or nonhuman serum (e.g., fetal calf or bovine serum) to the culture media. The concentration of serum required to derive the glial cells from the brain tissue will be in the range of about 0.1% to about 20% vol/vol. The glial cells, which may be astrocytes, oligodendrocytes, or microglia, may be normal or genetically engineered to secrete a therapeutic factor, the latter of which may be a neurotrophic factor and/or an immunomodulatory cytokine. Examples of such neurotrophic factors and/or immunomodulatory cytokines include, but are not limited to, insulin growth factor-1 (IFG-1), interleukin 10 (IL-10), and interleukin 13 (IL-13). The proportion of DA neurons to purified glial cells in the cell suspension may vary in proportion from about 0.01% to 99.99%, more specifically from about 1% to about 99. In an additional embodiment, decellularized extracellular matrix (ECM) derived from one or more glial or stem cell cultures may be grafted with DA neurons to enhance survival, differentiation, and innervation of the cell suspension comprising the DA neurons and purified glial cells. ECM derivation from glial or stem cell culture will be performed using standard chemical decellularization processes. The ECM generated can be used intact (Lyophilized) or it may be powdered into nanoparticies using high-pressure homogenization or cryogenic freezing. Either the intact or powdered ECM can be mixed or previously cultured of DA neurons prior to transplantation to the brain. In a further embodiment, exosomes may be added to the cell suspension. Exosomes may be prepared from conditioned media collected from one or more glial or stem cell cultures using standard exosome isolation reagents, which are commercially available (e.g., ThermoFisher Scientific, Waltham, Mass.) or by differential centrifugation. The exosomes can be either added directly to the cell suspension or added to the DA neuron culture prior to implantation into the brain.

In one embodiment, the neurological disorder is Parkinson's disease and a cell suspension of DA neurons and purified glial cells is implanted into one or more areas of the brain of the Parkinsonian patient: basal ganglia (in the forebrain); striatum (in the forebrain); and substantia nigra in the midbrain (which is characterized by dopamine cell loss in patients with Parkinson's disease). Target areas of the basal ganglia may include the putamen and the caudate. Simultaneous implantation into the striatum and substantia nigra may have the dual advantage of producing a protective effect on both DA neurons in the substantia nigra and dopaminergic terminals in the striatum.

In another embodiment, stereotaxic functional surgery is used to insert multiple needle tracks to deposit the dopaminergic cell suspension (comprising DA neurons alone or together with glial cells) into the putamen, the caudate and/or the substantia nigra. The stereotaxic surgical procedure may use a standard brain atlas or magnetic resonance imaging (MRI) scans to identify the coordinates of the target graft area. In a further embodiment, interventional intra-operative MRI is used to guide the injection of the cells into the target area in the brain. In practice, the dopaminergic cell suspension may be injected at a speed of 1 μl/min into the patient's brain with repeat injections occurring after a waiting period of 5 to 10 minutes after the last injection is finished. The number of needle tracks and/or repeat injections as well as the number of cells injected into the brain may vary from patient to patient depending on the degree of the patient's Parkinsonian symptoms and the area of denervation in the patient's brain.

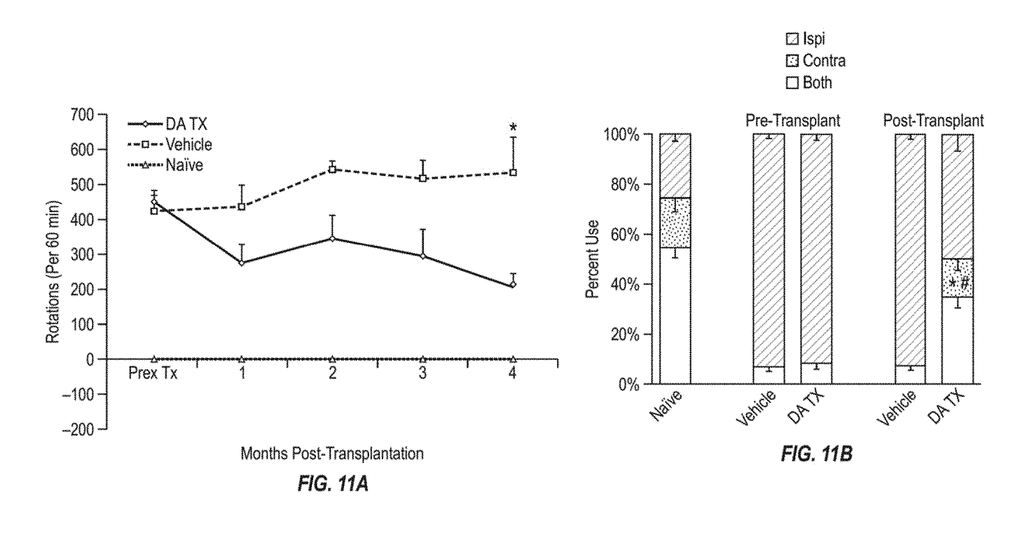

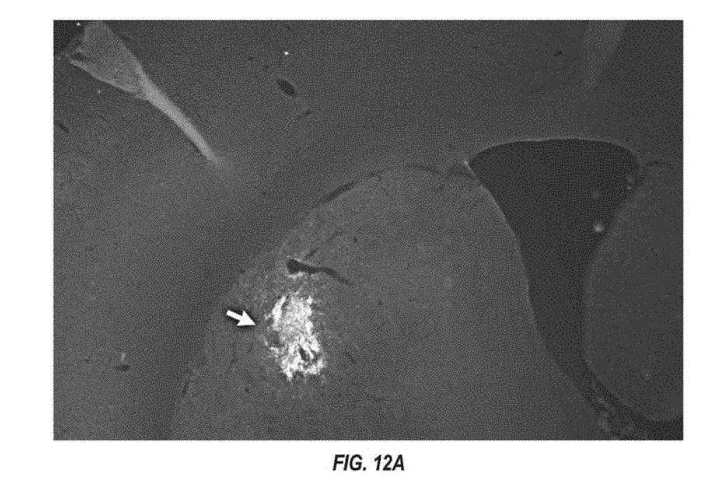

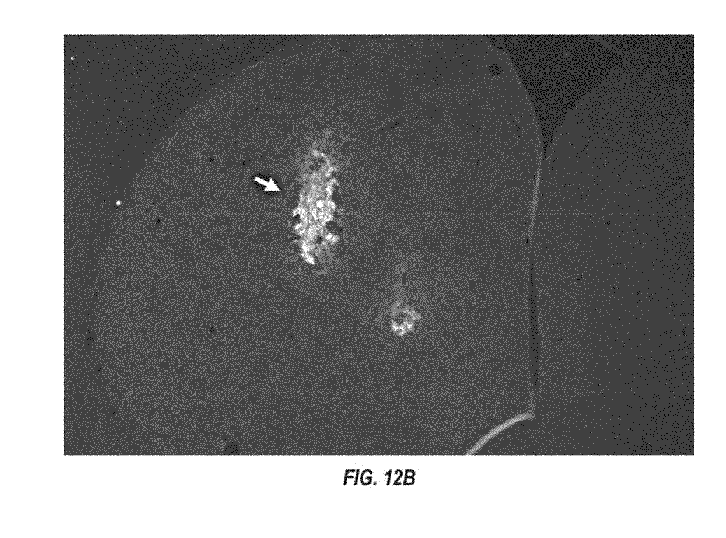

As shown and described in FIGS. 11A, 11B, and Example 3, the implantation of DA neurons into the striatum of an animal model of Parkinson's disease showed improved motor behavioral deficits. FIG. 11A shows the results of an apomorphine-induced rotational test in a rat with induced Parkinson's symptoms that underwent a DA neural cell implant (DA TX). The reduction in the rotations per minute in the rats treated with the DA neural cell implant demonstrates that DA neural cell implant had improved the Parkinsonian symptoms in the rats. FIG. 11B shows improved use of the contralateral forelimb (Contra) of a Parkinsonian rat that has been treated with a DA neural cell implant (DA TX). FIGS. 12A and 12B show low power microphotographs of frontal brain tissue sections of Parkinsonian rat brains grafted with DA neurons on the putamen and caudate of the striatum. Immunolabeling of the tissue sections with anti-TH antibody (a marker for DA neurons) shows the site of the DA neural cell implant (arrows).

All literature discussed in the Background of the Invention, the foregoing Detailed Description of the Invention, and the following Experimental are incorporated by reference into this patent or application.

### EXPERIMENTAL

The following procedures and tests were used to obtain the data provided herein. While efforts have been made to ensure accuracy with respect to variables such as amounts, temperature, etc., experimental error and deviations should be taken into account. Unless indicated otherwise, parts are parts by weight, temperature is degrees centigrade, and pressure is at or near atmospheric. All components were obtained commercially unless otherwise indicated.

The following procedures were used in the Examples or to obtain the data provided in the figures.

### Quantitative RT-PCR Analysis.

Quantitative real-time polymerase chain reaction (Q-PCR) was used to prepare the genetic data for FIGS. 2, 4, 6, and 8 and to test the NSC cells of Example 1. Total RNA was extracted from the NSCs at different stages using an RNeasy® kit (Qiagen GmbH, Hilden, Germany), Aliquots (1 μg) of total RNA from the cells were reverse transcribed in the presence of 50 mM Tris-HCl, pH 8.3, 75 mM KCl, 3 mM MgCl2, 10 mM DTT 0.5 μM dNTPs, and 0.5 μg oligo-dT (12-18) with 200 U Superscript RNase H-Reverse Transcriptase (Invitrogen, Carlsbad, Calif.). Quantitative real-time polymerase chain reaction (Q-PCR) using Applied Biosystems TaqMan® Gene Expression Assays (Roche Molecular Systems, Pleasanton, Calif.) was performed in the StepOnePlus® Q-PCR (Applied Biosystems, South San Francisco, Calif.) equipped with software for gene expression analysis. The expression of the gene of interest was determined in triplicate for each culture condition. Expression of the reference gene, 18S, was determined for each sample in triplicate. Quantification was performed at a threshold detection line (Ct). The Ct of each target gene product was normalized against that of the reference gene, 18S, which was run simultaneously for each marker. Data were expressed as mean ±SEM. The ΔCt for each candidate was calculated as ΔCt of [Ct(target gene)-Ct(18S)] and the ΔΔCt was the difference between Ct of the treated sample and the Ct of the control sample. The relative expression was calculated as the 2̂^{ΔΔC}^{ₜ} according to known methods and plotted as relative levels of gene expression. Livak & Schmittgen, Methods 35:402-408 (2001). For microarray analysis, the RNA extracted for the Q-PCR was subjected to Illumina® microarray analysis (Illumina, San Diego, Calif.). The data were transported into an Excel® spreadsheet (Microsoft, Redmond, Wash.), curated, and analyzed using GeneSpring® software (Agilent Santa Clara, Calif.) and Ingenuity® Pathway Analysis® gene analysis software (Qiagen, Redwood City, Calif.), the latter of which was used to prepare the schematics of FIGS. 3, 5, and 7.

### Immunocytochemistry.

The following Immunocytochemistry procedure was used to prepare the cell staining photographs of FIGS. 1A-1C, 2A, 2B, and 10A and in Example 4. Mouse monoclonal antibodies and polyclonal antisera were directed against neurotransmitter phenotypes, including GABA, substance P, NPY, somatostatin, met-enkephalin, and glutamate. The following neuronal antigens were used as primary antibodies for indirect immunofluorescence: polyclonal anti-tyrosine hydroxylase (1:1000; Millipore®/Chemicon®, Merck, Frankfurter, Germany) and anti-tubulin class III (monoclonal 1:1000, Sigma-Aldrich, St. Louis, Mo.). Coverslips were fixed with 4% paraformaldehyde for 20 minutes followed by three washes in PBS, 5 min each. Following the PBS rinse, coverslips were processed for dual labeling and incubated with the primary antibodies generated from different species that were added together in PBS/10% normal goat serum/0.3% Triton X-100 for 2 hours at 37° C. Following 3 rinses in PBS, secondary antibodies were applied in PBS for 30 min at room temperature and then washed three times (5 min each) in PBS, rinsed with water, placed on glass slides, and cover slipped using Calbiochem™ Flurosave™ (EMD Millipore, Hayward, Calif.) as the mounting medium.

### Example 1

### Isolation of Self-Renewable Neural Stem Cells and Preparation of Dopaminergic Neural Stem Cells

### Isolation of NSCs.

A human dermal skin biopsy was grown into fibroblasts, the Litter of which were transfected with a plasmid containing the Yamanaka factors OCT3/4, SOX2, KLF4, L-MYC, and LIN28. Okita et al., supra. Induced pluripotent stem cell (iPSC) colonies generated in the transfected fibroblast cultures were selected and expanded and neural stem cells (NSCs) were isolated from the iPSCs using serum-free media (1:1 mixture of Dulbecco's Modified Eagle's Medium (DMEM) and Ham's F12 nutrient (Thermofisher Scientific, Waltham, Mass.)) supplemented with 10-20 ng/mL of epidermal growth factor (EGF) (Millipore, Hayward, Calif.) and 10-20 ng/mL of human recombinant basic fibroblast growth factor (bFGF) (Millipore, Hayward, Calif.). Pluripotent colonies were perpetuated and used to isolate the self-renewable NSCs, which undertook multiple cell divisions, were passaged weekly, and remained stable and normal for more than 70 weeks. Retinoic acid was added to the NSC cell culture following the first week of cell division.

### Preparation of DA Neurons.

To differentiate NSCs into DA neurons, spheres of the self-renewing NSCs were collected and plated on poly-L-ornithine- or laminin-coated glass coverslips and treated with dopaminergic inducing media in order to subject the NSCs to dopamine-inducing conditions.

### Dopaminergic Inducing Media.

Dopamine inducing media was prepared from cultures of glial cells. Daadi & Weiss, The Journal of Neuroscience 19(11):4484-4497 (1999). The glial cells were cultured in DMEM/10% fetal bovine serum (FBS) until confluence at which tune the confluent glial cell cultures were rinsed once with phosphate buffered saline (PBS) and twice with serum-free DMEM/F12 (1:1) media supplemented with glucose (0.6%), glutamine (2 mM), sodium bicarbonate (3 mM), and HEPES buffer (5 mM), insulin (25 μg/ml), transferrin (100 μg/ml), selenium chlorine (30 nM), progesterone (20 nM) and putrescine (60 μM). After rinsing, the confluent cultures were mixed with 20 mL of the serum free media and placed in an incubator. The media was collected after 48 hours and centrifuged at 1000 g and 2000 g to remove cellular debris. The media was carefully removed, filtered, aliquoted, and stored at −80° C. The dopamine-inducing media included glial-secreted factors, bFGF, ascorbic acid, and retinoic acid. Other growth factors added to the media included a mix of human recombinant brain-derived neurotrophic factor (BDNF) and glial-derived neurotrophic factor (GDNF); human recombinant platelet-derived growth factor bb (PDGF-bb); FGF1; FGF2 (bFGF); FGF4; FGF7; transforming growth factors b2 and b3 (TGF-β2, TGF-β3); activin A (b-subunit); bone morphogenetic protein (BMP-2); human recombinant transforming growth factor a (TGF-α); rat recombinant ciliary neurotrophic factor (CNTF); Sonic hedgehog (Shh); and calcitonin gene-related peptide (CGRP).

### Example 2

### Preparation and Testing of Parkinsonian Rats

Adult Sprague-Dawley rats (2.75 g-310 g) were induced with Parkinsonism by unilateral injection of the neurotoxin 6-OHDA (also known as Oxidopamine) into the rostral part of substantia nigra pars compacta (SNpc). 6-OHDA kills DA neurons in the mid-brain and thus, mimics symptoms of Parkinson's disease in rats. All animals received 2 injections of 2 μl of 6-OHDA-hydrochloride (2 mg/ml in 0.2% ascorbic acid in sterile saline solution) first into SNpc at the stereotaxic coordinates: Anterior (A): 4.8 mm, Lateral (L): 1.9 mm and 7.1 mm ventral (V) from dura and second in the Medial Forebrain Bundle (MFB) at Posterior (P): 4.4 mm, L: 1.1 mm, V: −7.5 mm. The animals were evaluated for baseline behavior before and after the lesion and cell implantation.

### Rotational Behavior Test.

Rotational behavior is a drug-induced rotational behavioral test that is monitored in automated rotometer bowls of 28 cm diameter×36 cm high. Ungerstedt & Arbuthnott, Brain Res. 24:485-493 (1970). To reveal the dopamine misbalance between the two sides of the hemi-Parkinsonian rat brain, animals are injected with the dopamine agonist apomorphine (0.05 mg/kg, SC). Just after injection, the animals are placed into the test bowl and the number of rotations the rat makes either clockwise or counterclockwise is recorded over a test period of 60 min. The mean of net rotations are expressed as full body turns per hour contralateral to the lesion. Of the rats that were given the unilateral injection of 6-OHDA, only those Parkinsonian rats with a minimum number of 400 turn hour four weeks after the lesion were used For the forelimb symmetry test described below and the DA neuron transplantation procedure described in Example 3.

### Forelimb Symmetry Test.

The forelimb asymmetry test is also known as the cylinder test. Tillerson et al., The Journal of Neuroscience 21(12):4427-4435 (2001). For this test, a rat is placed in a transparent acrylic cylinder (20 cm diameter, 30 cm height) for 5 minutes. The cylinder encourages the use of the forelimbs for vertical exploration. The number of contacts on the wall during rearing is counted for etch paw. The data are presented as left paw contacts over right paw contacts and detect paw preferences. The forelimb Symmetry Test was performed on the Parkinsonian rats that had met the Rotational Test conditions specified above.

### Example 3

### Dopaminergic Neuron Implantation

iPSC-derived DA neurons were suspended at a concentration of 100,000 cells/μl in Isolyte® solution (B. Braun Medical Inc., Bethlehem, Pa.). The Parkinsonian rats of Example 2 were implanted with 1.5 μL of cell suspension at two striatal sites at the following brain coordinates (in mm): AP/ML/DV: +0.4/−3.0/−5 and −0.5/−3.6/−5. All animals Were immunosuppressed with cyclosporine A (IP, 20 mg/kg, Novartis) starting 1 day before surgery and continuing daily at 15 mg/kg per day. As shown in FIG. 11A, implantation of DA neurons into the brain of the Parkinsonian rats improved the motor behavior of the rats as exhibited by the decreased number of rotations per 60 minutes from 1-4 months following the DA neuron implantation versus the Parkinsonian rats that did not receive treatment with DA neurons (Vehicle). The graft of the DA neurons had reduced the amphetamine-induced rotation of the rats subjected to the rotation test. FIG. 11B shows that implantation of DA neurons into the brain of a Parkinsonian rat also improved the use of the rat's debilitated contralateral (Contra) forelimb during the lateral exploratory behavioral in the cylinder test (as the ipsilateral forelimb was not affected by the lesion, the ipsilateral forelimb did not show improvement).

### Example 4

### Immunohistochemistry Detection of Grafted DA Neurons in the Striatum

The Parkinsonian rats from Example 3 were euthanized at 4-months survival time by transcardial perfusion with phosphate buffered saline (PBS) followed by 4% paraformaldehyde. The brains from the animals were cryoprotected in an increasing gradient of 10, 20 and 30% sucrose solution and cryostat sectioned at 40 μm and processed for immunocytochemistry with anti-TH antibody. FIGS. 12A and 12B show that grafts of DA neurons immunostained with anti-TH inside the striatum (putamen and caudate) of a Parkinsonian rat brain.

I claim:

1. A method for isolating a population of neural stem cells from pluripotent stem cells comprising the steps of:
(a) obtaining pluripotent stem cells from embryonic stem cells obtained from an organism or inducing pluripotent stem cells from any somatic cell of a non-embryonic organism; and
(b) treating the pluripotent stem cells with epidermal growth factor (EGF) in a concentration in the range of about 10 ng/mL to about 100 ng/mL and basic fibroblastic growth factor (bFGF) in a range of about 10 ng/mL to about 100 ng/mL, wherein the pluripotent stem cells differentiate into self-renewable neural stem cells.

2. The method of claim 1, wherein the EGF and bFGF are both in a range of about 10 ng/mL to about 50 ng/mL.

3. The method of claim 1, wherein the EGF and the bFGF are both in range of about 10 ng/mL to about 20 ng/mL.

4. The method of claim 1(b), further comprising treating the pluripotent stem cells with retinoic acid having a molar concentration in a range of about 0.1 μM to about 10 μM.

5. The method of claim 1, further comprising treating the neural stem cells with retinoic acid within one week of isolation from the pluripotent stem cells.

6. The method of claim 1, where the self-renewable neural stem cells are passaged on a weekly basis.

7. The method of claim 1, wherein the self-renewable neural stem cells express at least one neural precursor marker.

8. The method of claim 7, wherein the at least one neural precursor marker is selected from the group consisting of nestin, vimentin, prominin-1, and Sox-2.

9. A method of treating a neurological disorder comprising administering a cell suspension of the neural stem cells of claim 1 to a patient suffering from the neurological disorder.

10. The method of claim 9, wherein the neurological disorder is selected from the group consisting of Alzheimer's disease, stroke, traumatic brain injury, amyotrophic lateral sclerosis, spinal cord injury, and Huntington's disease.

11. A method for inducing dopaminergic neurons from neural stem cells comprising treating a culture of the self-renewable neural stem cells of claim 1 with dopaminergic inducing media comprising at least one glial secreted soluble factor, wherein dopaminergic inducing media causes differentiation the self-renewable neural stem cells to differentiate into dopaminergic neurons expressing tyrosine hydroxylase.

12. The method of claim 11, wherein dopaminergic inducing media further comprises bFGF, ascorbic acid, forskolin, and optionally cAMP and/or retinoic acid.

13. The method of claim 11, wherein the at least one glial secreted soluble factor is present in the dopaminergic inducing media in a concentration ranging from about 25% wt/vol to about 75 wt/vol.

14. The method of claim 11, wherein the bFGF is present n the dopaminergic inducing media in a concentration ranging from about 10 ng/ml to about 100 ng/ml.

15. The method of claim 11, wherein the ascorbic acid is present in the dopaminergic inducing media in a molar concentration of about 1 μM to about 1 mM.

16. The method of claim 11, wherein the forskolin is present in the dopaminergic inducing media in a molar concentration of about 1 μM to about 1 mM.

17. The method of claim 11, wherein the culture of the self-renewable neural stem cells are further treated with an extracellular matrix or substrate selected from the group consisting of decellularized glial cells, decellularized neural cells, decellularized non-neuronal cells, poly-L-ornithine, fibronectin, and at least one laminin family member.

18. The method of claim 17 wherein the decellularized non-neuronal cells are choroid plexus cells.

19. The method of claim 17, wherein the poly-L-ornithine is present in the extracellular matrix or substrate in a concentration of about 1 ng/ml to about 50 μg/ml.

20. The method of claim 17, wherein the fibronectin is present in the extracellular matrix or substrate in a concentration of about 1 ng/ml to about 50 μg/ml.

21. The method of claim 17, wherein the at least one laminin family member is laminin-521 and/or laminin-511, wherein the laminin-521 and/or laminin-511 is present, individually or in combination, in the extracellular matrix or substrate in a concentration of about 1 ng/ml to about 50 μg/ ml.

22. The method of claim 17, wherein the extracellular matrix or substrate are added to the culture of self-renewable neural stem cells poor to treatment with the dopaminergic inducing media.

23. The method of claim 22, wherein the extracellular matrix or substrate is a solubilized mixture of protein added to the culture of self-renewable neural stem cells.

24. The method of claim 22, wherein the extracellular matrix or substrate coats a vessel upon which the self-renewable neural stem cells are cultured.

25. The method of claim 17, wherein the extracellular matrix or substrate is a solubilized mixture of protein added to the dopaminergic inducing media.

26. The method of claim 11, wherein the culture of the self-renewable neural stem cells are further treated with exosomes.

27. The method of claim 11, wherein the at least one glial secreted soluble factor is a TFG-β family member that activates a TFG-β signaling pathway.

28. The method of claim 27, where n the TFG-β signaling pathway is a Wnt/β-catenin pathway.

29. A method of treating a patient suffering from a neurological disorder associated with a loss or dysfunction of dopaminergic neurons comprising administering a cell suspension comprising the dopaminergic neurons of claim 9 to the patient, wherein the dopaminergic neurons improve symptoms of the disease.

30. The method of claim 29, wherein the neurological disorder is selected from the group selected from Parkinson's disease, trauma, bipolar disorder, depression, addiction, and schizophrenia.

31. The method of claim 29, wherein the cell suspension of dopaminergic neurons further comprises purified glial cells.

32. The method of claim 31, wherein the purified glial cells are selected from the group consisting of astrocytes, oligodendrocytes, and microglia.

33. The method of claim 31, wherein the purified glial cells are genetically engineered or processed to secrete neurotrophic factors and/or immunomodulatory cytokines.

34. The method of claim 33, wherein the neurotrophic factors and/or immunomodulatory cytokines are selected from the group consisting of insulin growth factor-1, interleukin-10, and interleukin-13.

35. The method of claim 31, wherein the dopaminergic/glial cell suspension have a proportion of dopaminergic neurons ranging from 0.01% to 99.99%.

36. The method of claim 29, wherein the cell suspension of dopaminergic neurons further comprises a decellularized extracellular matrix derived from one or more glial cell cultures.

37. The method of claim 30, wherein the neurological disorder Parkinson's disease.

38. The method of claim 37, wherein the cell suspension is implanted into basal ganglia forebrain areas of a patient with Parkinson's disease.

39. The method of claim 37, wherein the cell suspensions implanted into putamen and/or caudate areas of the basal ganglia of a patient with Parkinson's disease.

40. The method of claim 37, wherein the cell suspension is implanted into striatum forebrain areas of a patient with Parkinson's disease.

41. The method of claim 37, wherein the cell suspension is implanted into substantia nigra midbrain areas of a patient with Parkinson's disease.
